(19) United States
(12) Patent Application Publication
(10) Pub. No.: US 2026/0013792 A1
(43) Pub. Date: Jan. 15, 2026

(54) SYSTEMS AND METHODS FOR SMART ATHLETIC WEAR

(71) Applicant: Wearable Experiments Inc., New York, NY (US)

(72) Inventors: Billie Josephine WHITEHOUSE, New York, NY (US); Benjamin James MOIR, Darlinghurst (AU)

(21) Appl. No.: 19/046,444

(22) Filed: Feb. 5, 2025

Related U.S. Application Data
(63) Continuation of application No. 16/068,314, filed on Jul. 5, 2018, now abandoned.
(60) Provisional application No. 62/275,232, filed on Jan. 5, 2016.

(51) Int. Cl. A61B 5/00; A41D 1/00; A61B 5/11; G06F 3/00; G06F 3/01; G06F 3/0346; G09B 19/00; G16H 20/30; G16H 40/63; G16H 40/67

(52) U.S. Cl. CPC A61B 5/6804; A41D 1/002; A61B 5/1121; A61B 5/486; G06F 3/00; G06F 3/016; G06F 3/017; G06F 3/0346; G09B 19/0038; G16H 20/30; G16H 40/63; G16H 40/67; A41D 2600/10; A61B 5/7455; A61B 2562/0219

(57) ABSTRACT

Embodiments of the current disclosure are directed towards a smart garment comprising one or more sensors contained within the smart garment and configured to gather information about a wearer of the smart garment undertaking a physical activity. The garment may also include one or more actuators contained within and configured to provide haptic feedback to the wearer of the smart garment. The one or more sensors are configured to transmit the gathered information to a processing unit within the smart garment and/or an external processor for generation of instructions to provide guidance to the wearer of the garment on how to execute the physical activity. Further, the one or more actuators can provide haptic feedback to the wearer upon receiving the instructions from the processing unit and/or the external processor.

### CROSS REFERENCES TO RELATED APPLICATIONS

This application is a continuation of U.S. patent application Ser. No. 16/068,314, filed Jul. 5, 2018, entitled “Systems and Methods for Smart Athletic Wear”, which is a national stage entry of, and claims priority to, International Patent Application No. PCT/US2017/012377, filed Jan. 5, 2017, entitled “Systems and Methods for Smart Athletic Wear,” which in turn is a non-provisional of and claims priority under 35 U.S.C. § 119 to U.S. provisional patent application Ser. No. 62/275,232, filed Jan. 5, 2016, titled “Systems and Methods for Smart Athletic Wear.” which is The disclosures of each of the above-referenced applications are expressly incorporated herein by reference in its entirety their entireties.

### FIELD OF THE DISCLOSURE

Embodiments of the current disclosure are directed toward systems and methods for smart athletic wears. In particular, the present disclosure relates to systems and methods for implementing smart capabilities in athletic wearables, including configurations for providing haptic feedbacks to users of the athletic wearables in real-time.

### BACKGROUND

During physical activities, and in particular those of the athletic nature, individuals partaking in the activities prefer to wear athletic wears that are specifically designed for the physical activity. For example, a same individual may wear a pair of yoga pants to yoga sessions, tennis shorts to tennis games, football jerseys to football (i.e., soccer) games, and bike shorts to bike rides. In some of these cases, the athletic wears may be in direct and continuous contact with the body of the wearer. In some embodiments, athletic wears designed for specific activities can be used to address physiological needs that naturally follow the particular physical activity. For example, athletic shirts designed to be worn during active sports or games such as running, football, etc., may be designed to allow efficient cooling of the wearer of the shirts as the wearer perspires during the activity.

### SUMMARY OF SOME OF THE EMBODIMENTS

Some embodiments of the present disclosure are directed towards a smart garment. In some embodiments, the smart garment comprises one or more sensors contained within the smart garment and configured to gather data on a stance and/or a movement of a wearer of the smart garment undertaking a physical activity; and one or more actuators contained within the smart garment and configured to provide haptic feedback to the wearer of the smart garment. The one or more sensors can transmit the data to a processing unit for generation of instructions providing guidance on executing the stance and/or the movement; and the one or more actuators can provide haptic feedback to the wearer upon receiving the instructions from the processing unit. In some embodiments, the smart garment can be a pair of yoga pants.

In some embodiments, the one or more sensors comprise an accelerometer, and/or a gyro sensor. Further, the one or more sensors may include at least two sensors, and the at least two sensors are configured to communicate with each other. In addition, the smart garment may comprise a processing unit disposed within the smart garment. In some embodiments, the processing unit may be disposed in a smartphone, and the above-noted instructions are displayed to the wearer via a user interface of the smartphone.

In some embodiments, the smart garment may further comprise a power source for providing power to electrical components contained within the smart garment. Further, the smart garment may include a communications circuit configured to facilitate communication amongst the one or more sensors, the one or more actuators and/or the processing unit. The communications circuit may further be configured to facilitate communication between electronic components contained within the smart garment and an external processor.

In some embodiments, the one or more actuators may be configured to vary one or more of an intensity, frequency and pattern of the haptic feedback based on content of the received instructions.

In some embodiments, the gathered data on a stance and/or a movement of a wearer of the smart garment may include parameters related to relative positional relationship of the one or more sensors with respect to each other during an execution of the stance and/or the movement by the wearer. For example, the processing unit can generate the instructions after comparing the parameters of the relative positional relationship of the one or more sensors to standard parameters representing accurate execution of the stance and/or the movement. The parameters of the relative positional relationship may include orientations of any two sensors with respect to each other. In some embodiments, the parameters of the relative positional relationship may include distance between any two sensors with respect to each other. In some embodiments, the smart garment may further comprise a timer for recording timestamp measurements during an execution of the stance and/or the movement by the wearer.

Some embodiments of the present disclosure are directed towards a system that comprises a smart garment and a communications circuit, wherein the smart garment includes a sensor and an actuator, the sensor configured to collect data on a stance and/or a movement of a smart garment wearer and the actuator configured to provide haptic feedback to the smart garment wearer in response to an instructions message received from a processing unit. The communications circuit, operationally coupled to the sensor and the actuator, may be configured to transmit: (1) the data collected by the sensor to the processing unit, and (2) the instructions message generated by the processing unit to the actuator. In some embodiments, the processing unit, upon receipt of the data from the communications circuit, may be configured to: identify a defining parameter of the stance and/or the movement of the garment wearer; extract, from the transmitted information, a value representing the identified defining parameter of the stance and/or the movement; and evaluate the extracted value for congruence with a standard value of the defining parameter of the stance and/or the movement, the standard value representing a correct execution of the stance and/or the movement.

In some embodiments, the sensor comprises a plurality of sensors, and the defining parameter includes orientation of any two sensors of the plurality of sensors with respect to each other. In some embodiments, the defining parameter includes distance between any two sensors of the plurality of sensors. In some embodiments, the system comprises a timer for recording timestamp measurements during an execution of the stance and/or the movement by the wearer.

Some embodiments of the present disclosure are directed towards a method comprising the steps of receiving, at a user interface of a computing device, a selection identifying a routine of a physical activity to be reenacted by a user wearing a smart garment, the routine characterized by a defining parameter; receiving, at a processor of the computing device, data on a stance and/or a movement of the user reenacting the routine, the data collected by a sensor integrated into the smart garment; extracting, from the received data, a value representing the defining parameter of the stance and/or the movement of the routine reenacted by the user; and evaluating the extracted value for congruence with a standard value of the defining parameter of the routine, the standard value representing a correct execution of the routine.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIGS. 1A-B show example placement of electronic components including actuators and sensors on various positions on a pair of athletic yoga pants, according to some embodiments.

FIG. 2 shows example schematic illustration of the interplay of a smart garment worn by a user, an external device such as a smartphone used by the garment wearer to facilitate the execution of a physical activity, and an external server, according to some embodiments.

FIG. 3A shows example yoga poses. FIG. 3B shows an example illustration of a feedback on the execution of a particular pose by a smart yoga pants wearer that may be provided by a smartphone to the user after an analysis of the user's execution based on data obtained from sensors onboard the yoga pants, according to some embodiments.

FIG. 4 is an example flow diagram showing the execution of a physical activity routine with guidance from an external device such as a smartphone by a wearer of a pair of smart athletic yoga pants, according to some embodiments.

### DETAILED DESCRIPTION OF SOME OF THE EMBODIMENTS

Some embodiments of the present disclosure are directed towards a smart garment comprising one or more sensors contained within the smart garment and configured to gather information about a wearer of the smart garment undertaking a physical activity. The garment may also include one or more actuators, audio devices and display components contained within and configured to provide haptic, vocal and visual feedback to the wearer of the smart garment. The one or more sensors are configured to transmit the gathered information to a processing unit within the smart garment and/or an external processor for generation of instructions to provide guidance to the wearer of the garment on how to execute the physical activity. Further, the one or more actuators, audio devices and display components can provide feedback to the wearer upon receiving the instructions from the processing unit and/or the external processor.

In some embodiments of the present disclosure, garments that are configured to sense various information about the wearer of the garments and communicate the data internally and/or externally are disclosed. In some cases, these garments which can be athletic wears may be skin tight, or may be designed to have at least substantial contact with the skin of the wearer. For example, yoga pants can be designed to be skin-tight, and the proximity of the yoga pants to the skin may facilitate the integration of various sensors and active elements such as actuators into the yoga pants so as to sense and gather information about, and interact with, the wearer of the pants, respectively. In some implementations, the sensors can be flexible so as to tightly follow the contour of the skin to which they are in close proximity. In some implementations, the sensors may be coupled to or include flexible circuit boards (in such cases, the sensors themselves may be flexible or non-flexible).

In some instances, the garments may contain sensors configured to capture data related to the wearer and/or the wearer's environment, and such sensors may communicate the sensed data to other sensors or electronic components such as processors in the garments and/or external devices such as smartphones via a communications component (e.g., wireless) incorporated into the garment. In some embodiments, the garments can also be configured to provide haptic, visual and audio feedback to the wearer. The feedback messages may be received from external devices such as smartphones, and/or may be generated from a processing unit in the garment itself. As an example, a haptic feedback may be in the form of a vibration induced by an actuator embedded in the garment, and the vibration may have been generated in response to a message from an external device and/or a processor. A visual feedback may include light patterns coming from LEDs, and different light patterns that are generated in response to a feedback message from a garment's processing unit and/or an external processor may represent different feedbacks (e.g., for a wearer of a yoga pants, the light of an LED being on, off or blinking may indicate whether or not the wearer has succeeded in attaining proper yoga poses or posture). Similarly, an audio feedback may be generated by a speaker in response to a feedback message from the processing unit of the garment and/or an external processor. In some embodiments, the external processor may be the processor of the external device (such as a smartphone the smart garment wearer is utilizing in executing a physical activity, for example) or an external server.

Examples of smart athletic wears into which the noted sensors, communication components, actuators, power sources, processors, etc. can be incorporated include shirts, jerseys, shorts, shoes, underwear, pants (e.g., yoga pants), and/or the like. For example, fan jerseys designed to simulate the effects of the activities of a physical sport to the wearer of the fan jersey are discussed in PCT/US/2014/072750, filed Dec. 30, 2014, entitled “System and Method for Effecting a Physical Experience,” the entire contents of which is incorporated by reference herein in its entirety.

In some embodiments, sensors embedded within the garment may measure and/or gather data related to the wearer of the garment and/or the environment. For example, GPS devices, accelerometers and/or gyro sensors in the garment may provide information on the location, orientation and/or motion of the garment/garment wearer, and in some instances, the spot on the garment at which the sensor is located. For example, if a gyro sensor located at the left knee of a yoga pant worn by a user provides a data set related to its orientation, this information may be viewed to apply to the left knee and not necessarily to the body of the wearer, since the wearer may be facing a different direction than the left knee. However, if the information is a location information obtained from a GPS device located at the left knee, the location information may also be ascribed to the wearer as a whole. In some embodiments, sensors may comprise clocks for measuring time and/or time durations. Health meters or detectors may monitor physiological or health related parameters of the wearer, in particular, those related to the physical activities of the wearer. For example, health-related sensors may monitor heart rates, perspiration levels, muscle contractions, body temperature, and/or the like.

In some embodiments, the data measured and/or gathered by the sensors may be communicated to a processing unit contained within the garment and/or to an external computer or server via a wired and/or wireless communications circuit. For example, the communications circuit may be a wireless communications chip operating in one or more of the communication protocols such as, but not limited to, the internet, Bluetooth, Bluetooth Low Energy (BLE), near field communication (NFC), Wi-Fi™, or any other radio frequency (RF) protocol. The communications circuit may also relay received messages to the sensors, visual components and audio devices, and/or actuators located in the garments. For example, the communications circuit may receive instructions from a processor embedded in the garment, and/or an external device such as a smartphone, to activate and cause the actuators to vibrate, which may then be relayed to the actuators. Similarly, the circuit may receive messages directed to the sensors (e.g., instructing the sensors to initiate or cease sensing, providing the sensors with sensing parameters, etc.), which it may then relay to the intended sensors. In the embodiments where the communications circuit receives instructions or messages from external processors that are external to a garment, the circuit may forward the instructions or messages to one or both of the processing unit and the intended recipient (whether sensors, actuators, electrical components, and/or the like). In some embodiments, garments worn by different users may communicate with each other via each garment's communications circuits. For example, the garments may communicate in a “master-slave” type arrangement, where one or more of “master” garments transmit messages/instructions to other garments. One example of such arrangements, discussed in more details below, is a yoga instructor-students setting, where various yoga poses as executed by one or more instructors are transmitted from the garments worn by the instructors to the garments worn by the students.

In some embodiments, the processing unit may comprise a processor (e.g., ARDUINO processor) configured to receive data from sensors or other processors and analyze the received data so as to determine the stance of the garment wearer. For example, the processing unit may receive sensor data on the orientation, height, motion, etc., of the one or more sensors in the garment, the sensors including one or more accelerometers and gyro sensors located at various points in the garment. In some instances, the processing unit may determine from the received data information on the positional relationship of the sensors with each other, such relationship including distance and orientation between sensors. In some embodiments, the garment may also contain sensors equipped with GPS capabilities, and the data received by the processing unit may also include location information. The collected data may also be time referenced, and as such, the processing unit may resolve the garment wearer's movements as a function of time progression. In some embodiments, the processing unit may analyze one or more of the above-noted data sets to establish the garment wearer's stance and movements, and use the results of the analysis to determine the accuracy and efficiency of the garment wearer's physical activities. For example, if the wearer is wearing a pair of yoga pants containing a plurality of sensors distributed over the pants, time-referenced or otherwise data on orientation, motion, location, relative displacement (of sensors with respect to each other, for example), etc., received from such sensors can be used by a processing unit to recreate the wearer's yoga poses and the transitions between them. Accordingly, the processing unit may, for example, determine if the wearer is performing the various yoga poses accurately by comparing the wearer's recreated movements and poses against “standard” movements and poses. The “standard” movements and poses may be pre-stored in a memory also embedded in the garment.

For example, if a yoga pose requires a left knee to be bent about 90°, a processing unit may calculate the angle the wearer's left knee is bent from some or all of the data received from the location/orientation sensors situated on various locations on the garment, including sensors located at or in the vicinity of the garment wearer's left hip, left knee and left ankle. The calculation may determine the angle, the uncertainties in the calculation, the offsets, if any, of the sensors from their expected positions, and in general any relevant determination pertaining to the positioning of the leg. In some embodiments, the processing unit may compare these determinations with some standard values stored in a memory to ascertain whether the garment wearer has assumed the correct position and/or executed the correct movements with respect to the yoga pose. In such embodiments, the processing unit may also generate instructions configured to cause one or more actuators, audio or visual devices to provide feedback to the user so as to attain the correct position (e.g., a specific pattern of vibration by an actuator may inform the garment wearer to lower her/his hips).

In some embodiments, the aforementioned determination of a garment wearer's activities (e.g., movements, poses, etc.) based on sensor data may also be performed by an external device such as a smartphone. In some embodiments, the processing unit may also transmit the received data and/or its determinations to the external device (via the communications circuit, for example). For example, an application executing on the smartphone may perform similar calculations as described above with respect to the processing unit abroad the garment to determine how accurately the garment wearer is performing the physical activities (by comparing the calculations with some standard values, for example). In some embodiments, the smartphone may transmit messages back to the garment (e.g., to the processing unit, sensors, actuators, etc., via the communications circuit, for example), with the messages being dependent on the smartphone's determination. For example, the message generated by the smartphone may instruct an actuator located at the ankle of the garment to vibrate in a manner that informs the garment wearer to shift her/his ankle so as to perform the activity accurately (e.g., attain the accurate yoga posture). The relationship between the vibration types (e.g., the vibration's intensity, pattern, duration, etc.) and the information intended to be transmitted to the wearer by the message (e.g., shift or rotate one's foot left or right, for example) may be pre-set and known by the garment wearer. In some implementations, the message may be relayed to the wearer via the audio and/or visual devices. In some embodiments, for the smartphone to issue instructions to the garment, or in general to be linked to and be recognized by the garment (more particularly, by the electronic components residing in the garment), it may be authorized beforehand by the garment wearer.

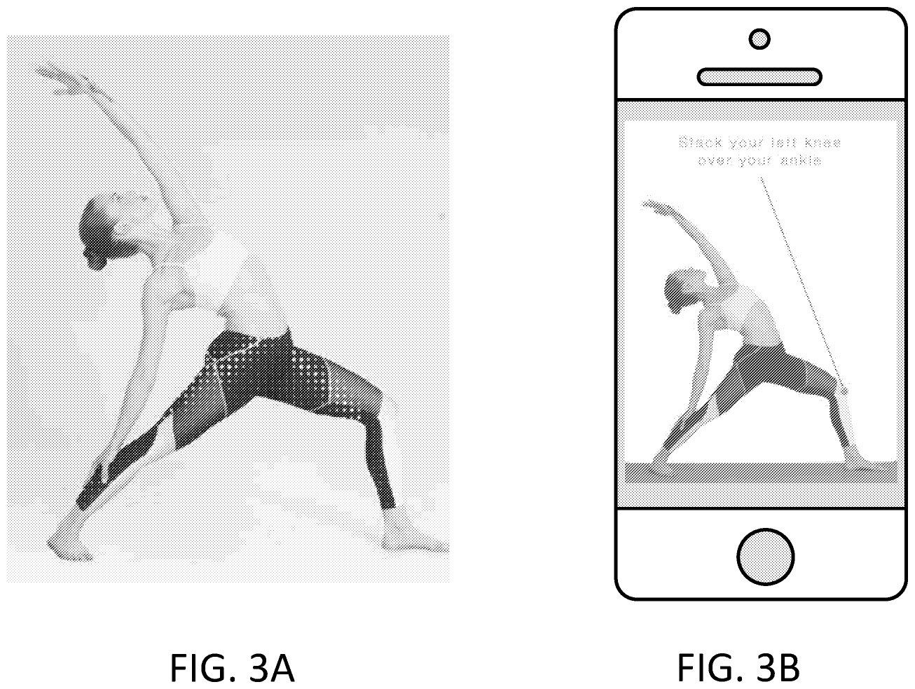

In some embodiments, a processing unit or an external processor may determine the accuracy and/or efficiency of the execution of a certain stance or physical activity by comparing the parameters defining the stance or physical activity to standard values of said parameters stored in a memory. The standard values or parameters that define the correct form may be stored in a memory embedded in physical garments and/or a memory of the external computer or server such as the smartphone (e.g., the smartphone authorized to be linked to the garment). For example, a yoga pose may comprise one leg straightened out backwards and the front of the leg making x° with the ground while the front of the other leg making y° with respect to the ground with a z° bent at the knee, and these parameters (the values for x, y and z, for example) may define the particular yoga pose or posture. An example of such a yoga pose is shown in FIG. 3A. In some embodiments, additional or alternative parameters may also be used to define same poses. For example, instead of or in addition to the angles, distances between sensors located at both the right and left hips, knees and ankles of a user's yoga pants may be used to define the noted pose. It is to be understood that other combinations of parameters may be used to define a yoga pose, including distances between sensors, height of sensors from the ground, orientations of sensors with respect to each other, and/or the like. In some embodiments, these parameters may depend on other variables. For example, these values may depend on variables related to the garment wearer, such as weight, height, etc. Such variables may be entered into the smartphone by a user, or the application executing on the smartphone may obtain such data from other sources (e.g., a health app running on the smartphone) such that the parameters may be updated based on the variables. For example, a parameter that is related to a distance between two sensors may be dependent on height as a variable as the parameter would be larger for a taller person in comparison to the same parameter for a shorter person.

In some embodiments, the standard values or parameters stored in the memory may be classified according to one or more settings based on some selected classification parameter. For example, the classification parameter may be expertise and the settings may range from “novice” to “expert”. Referring to the above example, an “expert” setting may be a setting where a user deviates from x°, y° and z° by at most a small amount while the deviations for intermediate and novice are progressively larger. As such, when a user selects the novice setting for a particular yoga pose and a processing unit determines, based on data sensed and transmitted by one or more sensors located on the yoga pants of the user, that the angles deviate from x°, y and z° by an amount too large for an expert setting but smaller than the acceptable deviation for a novice, the processing unit may determine that the user has achieved a correct stance for the yoga pose. If, instead, the user had chosen the expert setting, the processing unit may then have determined that the user's pose was not correct, and in some instances, either notify the user and/or log the determination into a log book for future review. The notification to the user may be in the form of a notification appearing on the user's smartphone, and/or a message to one or more of the haptic actuators, audio devices and visual displays embedded or integrated into the yoga pants of the user to inform the user of the determination by the processing unit (e.g., the haptic actuators at one of the left and right hips may vibrate in a manner that indicates to the user to lower the hip so as to reduce the large deviations from x°, y° and z°). It is to be understood that the above embodiment is for illustration purposes, and any manner of defining a stance or movement, determining if the correct stance or movement is achieved, and communicating the determination to the use via an external device such as a smartphone or actuators and other communication devices onboard the garment are contemplated to be within the scope of the current disclosure. Further, it is to be understood that although yoga pants are discussed as an example, other smart wearables such as shirts, shoes, etc., may be used in a similar manner to monitor the physical activities of the wearer of the smart wearable and provide feedback and/or information to the wearer on how to improve execution of the physical activities.

In some embodiments, for example when a user of a smart garment wishes to use the smart garment during a physical activity, the user may utilize an external device such as a smartphone to communicate with one or more of the electrical components onboard the garment. In some embodiments, the wearer of the smart garment may activate or power on the electrical components in the garment, and establish a link between the garment and a smartphone. For example, a connection may be established between the communications circuit in the smart garment and the smartphone, the connection being in the form of one or more of the communication protocols such as, but not limited to, the internet, Bluetooth, Bluetooth Low Energy (BLE), near field communication (NFC), Wi-Fi™, or any other radio frequency (RF) protocol. An application executing on the smartphone may be used to communicate with and in general control the sensors and/or the actuators embedded in the smart garment. For example, as discussed in some detail above, a processor in the external device may determine from received garment sensor data on whether a user's pose has achieved the correct form or not (according to a standard stored in a memory of the device, for example), and generate and transmit informative/instructive messages to the electrical components (via a communications circuit on the garment, for example).

Upon the establishment of a connection between the smartphone and the smart garment, in some embodiments, the smart garment may be synchronized with the smartphone, and the application may avail for a user's selection one or more routines of the physical activity that is associated with the smart garment. For example, upon synchronization of a pair of smart yoga pants with a smartphone, an application executing on the smartphone may reveal one or more routines comprising yoga poses for the smart garment wearer's selection. In such embodiments, a routine may be selected by the garment wearer (or someone else on the garment wearer's behalf), and instructions may appear on the smartphone providing guidance to the user on how to execute the routine. Further, instructions may be sent to the actuators in the smart garment so as to cause the actuators to vibrate in manners indicating the execution of the routine. In some embodiments, if the smart garment wearer is not executing the routine accurately, the smartphone may generate further instructions to correct the inaccuracies in the wearer's routine or poses as described above. In addition, the smartphone may keep logs of the wearer's activities and/or errors for display on the user interface of the smartphone and/or a later review.

For example, a user wishing to perform one or more yoga poses may establish a connection and synchronization with an app executing on a smartphone, the app configured to avail to the user at least one yoga pose routine to emulate. Upon selection of at least one routine, in some embodiments, the sensors may transmit sensed data to the smartphone or the processing unit on the yoga pants via a communications circuit on the yoga pants. For example, there may be sensors located at various points of the yoga pants, including at the hips, back of knees and ankles of both legs of the yoga pants wearer. The data gathered by these sensors, which may comprise one or more of gyroscopes, accelerometers, GPS devices, clocks, and/or the like, may include positional and temporal information related to the user's yoga pose, including the locations and orientations of the sensors at various times during the movement and stance of the user. The locations data may be with respect to a reference point or a plane (e.g., height as measured with respect to the ground, etc.), with respect to each other (e.g., distance between any two sensors), and/or the like. Similarly, the orientations may be with respect to some defined plane (e.g., the user body's sagittal, coronal and/or axial planes). In some embodiments, the sensors can be flexible, and as such may closely follow the contour of the skin to which they are coupled, whether directly or indirectly (e.g., through a clothing).

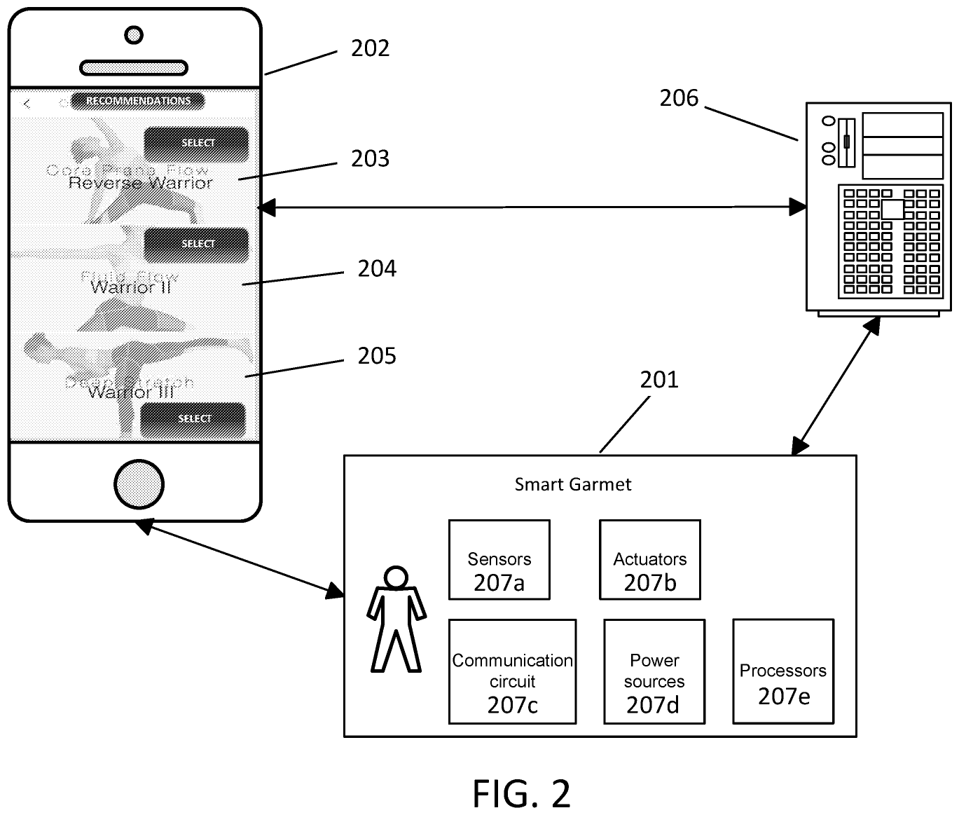

Upon receiving the sensed data, the processing unit and/or the processor on the smartphone may then perform calculations and/or comparisons to establish if the user's execution of the pose has been accurate. An example of the type of analysis that would be performed by a processor has been discussed above with respect to an example embodiment. As a summary, the processor, upon receiving the data, may compare the data and/or results obtained by further calculations involving the data (e.g., if the data includes the locations of two sensors, the processor may calculate the distance between the sensors, etc.). The processor may then select from the received data and/or the calculated results the parameters that define the particular pose. For example, if the yoga pose is the Warrior I pose (other poses are shown in FIGS. 2 and 3), the processor may select from the received data/calculated results the location data of the sensors at the knees and ankles as parameters for comparison with standard values. This follows because for the Warrior I pose, the knee and ankle locations of both legs could be used as standard parameters since, when striking the pose, the knee and ankle sensors on one leg have to align vertically while the same sensors on the other leg should not align vertically. Accordingly, the processor may then compare the vertical alignments of the knee and ankle sensors of each leg as measured by the received data/calculated results to those of the standard parameters (e.g. those stored in a memory on the garment and/or smartphone), and based on the comparison, may establish whether the user has achieved an accurate pose or not.

In some embodiments, if the processor establishes that the user has not achieved the correct pose, the processor may then investigate the cause of the failure to achieve the correct pose and determine a correction that may lead to success. With respect to the foregoing example, the processor may determine, based on the received data/calculated results, that the knee bent angle of the leg (with the sensors that were supposed to be vertically aligned) deviates significantly from about 90°, resulting in the vertical misalignment of the knee and the sensors. With such determination, the processor may then generate a message for transmission to one or more of the actuators, audio devices and visual elements to inform the user to stack the misaligned knee above the ankle so as to achieve an acceptable alignment. For example, the processor may generate a message that, upon receipt by an actuator located at the knee, vibrates in a manner that instructs the user to bend the knee to about 90°. The user may deduce the information from the vibration based on any number of vibration characteristics, such as duration, intensity, pattern, etc. Similarly, a visual element such as a light source may blink correspondingly to inform the user of the same information, and an audio device may vocally relay the information to the user. In some embodiments, the same information may be displayed on the display of the smartphone.

As discussed above, in some embodiments, the standard parameters may be different depending on the setting selected by the user (or by default) for the user's physical activity. For example, with reference to the above example, an “expert” setting may require no more than a 3° deviation from perpendicular knee bent for the pose to be considered accurate by the processor. On the other hand, a larger deviation such as 10° maybe be acceptable for a “novice” expertise setting.

In some embodiments, the processor may still generate and transmit a message to the smart garment of the user even when the determination indicates that the user has executed a correct pose. For example, the message may be an encouragement or a confirmation of the successful pose.

In some embodiments, in addition to or instead of the parameters defining the correct form being obtained from memories in the smart garment and/or the external device, the parameters may be generated by another garment in real time. For example, a person performing a physical activity may be wearing a first smart garment, which may be linked to an external server and/or one or more second smart garments worn by other users. The second smart garments may also be linked to the external server. The connections or links may be in the manner of one or more of the communication protocols such as, but not limited to, the internet, Bluetooth, Bluetooth Low Energy (BLE), near field communication (NFC), Wi-Fi™, or any other radio frequency (RF) protocol.

In some embodiments, when the person donning the first smart garment performs a physical activity, the data measured and/or gathered by the sensors embedded in the first smart garment may be recognized as representing a correct form of performing the physical activity. For example, the person wearing the first garment may be a yoga instructor, and when the person assumes a certain yoga posture or pose, the parameters sensed by the sensors in the first garment (e.g., orientations, relative location (e.g., distance and/or orientation between the various sensors), etc.) may be transmitted to the external server and/or the processing units of the one or more second garments (pairs of yoga pants worn by students, for example) as parameters of the correct form the wearers of the second garment should assume. Accordingly, in such embodiments, when the posture of a wearer of a second garment does not conform to the parameters generated in real time by the first garment and transmitted to the external server and/or the second garment(s), the processing units in the second garment(s) and/or the external server may perform similar calculations and/or comparisons as described above for determining whether a wearer of a smart garment has achieved a correct pose (e.g., similar to the discussion above with respect to the Warrior I yoga pose). Upon the determination, the processing units of the second garments and/or the processor of the external device may generate instructions for transmittal to the one or more actuators, audio elements and visual displays in the second garment. For example, with respect to the actuators, the instructions may be configured to cause the one or more actuators in the second garments provide feedback to the wearers of the second garments to correct the form of their pose.

In some embodiments, the sensors, power sources (e.g., battery packs), processing unit, communications circuit, actuators (e.g., touch actuators), and any other electronic unit, etc. can be located throughout a smart athletic garment, the locations of the components strategically selected based on a variety of factors. For example, with respect to the actuators, that the placement of the actuators may be selected so as to allow the garment wearer to feel the vibrations of the actuators well enough to recognize the information meant to be delivered by the vibrations. For example, the actuators may be situated in close proximity to the user's skin such that the user readily identifies the pattern, duration, intensity, etc., of the vibrations. Other examples of selection factors for placing actuators in garments include aesthetic preferences (for example, locations may be in less visible spots (inner seams of yoga pants or incorporated within a decoration for yoga pants, for example)), sturdiness (inner seams may provide sturdier “packaging” for electronic components), safety reasons, etc. For example, a battery pack and the electronic components (e.g., processing unit, communications circuit) may be located behind a knee. In some embodiments, the components may be removably incorporated and/or permanently integrated into smart garments. In some embodiments, the actuators may be connected to processing units onboard the garment.

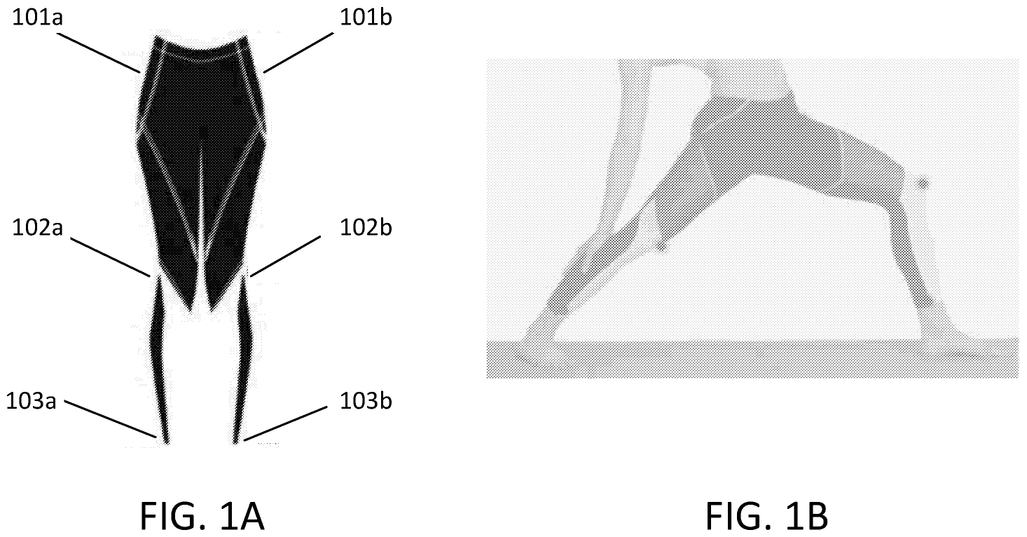

With reference to FIGS. 1A-B, in some embodiments, front, side and back views of a schematic design of a pair of smart yoga pants are shown. The disclosed pair of smart pants may comprise a main body, which may or may not include a waistband, trimmings, and one or more functional pieces. The main body may be form fitting in nature, made of fabrics such as bamboo spandex fabrics. The waistband may comprise elastic materials configured to hold the smart pants on when worn by the user. The waistband may range in width from about 1 mm to about 10 mm, from about 2 mm to about 9 mm, from about 3 mm to about 8 mm, from about 4 mm to about 7 mm, from about 4.5 mm to about 6 mm, about 5 mm and/or the like. The one or more functional pieces may include may be configured to enhance one or more of air circulation and aesthetic appeal of the yoga pants. For example, a functional piece may comprise a mesh fabric, the mesh ranging in size from about 4-mesh to about 200-mesh, about 12-mesh to about 140-mesh, about 30-mesh to about 100-mesh, about 60-mesh to about 80-mesh, including all values and ranges therebetween.

FIG. 1A shows the placement of electronic components on various positions on a pair of athletic yoga pants are shown. Electronic components such as but not limited to sensors, actuators, communications circuit, drivers, power sources (e.g., battery) and/or the like may be placed at any position on the smart pants, and one or more positions may be selected based on manufacturer and/or user preferences with respect to aesthetics, safety, ease of use, use of manufacturing, and/or the like. These electronic components may be flexible, and as such may be configured to adapt to the shape of the skin with which the components are directly or indirectly contacted. Further, in particular with respect to sensors and/or actuators, the numbers and locations of the positions may be selected so as to generate at least adequate data points for determining a desired level of the physical activity of the smart pants wearer. In some embodiments, pockets may be positioned at select locations on the smart pants so as to serve as receptacles or housings for the electrical components. In some embodiments, the electrical components may be seamlessly integrated into the fabric of the yoga pants. In such embodiments, the integration may be removable and/or permanent.

Examples of locations on the smart yoga pants at which electronic components can be situated include the hips 101a and 101b, back of the knees 102a and 102b, ankles 103a and 103b, traditional front pocket locations, traditional back pocket locations, at the waistband along the waist (e.g., at the sacrum (middle of the lower back) and around the hips in either direction), along any seams of the smart pants, and/or the like. As discussed above, at these and other locations for placing the electronic components, there may be pockets or housings for receiving the components and/or the components may be integrated into the fabrics. FIG. 1B shows schematically example placements of sensors on a pair of yoga pants worn by a user that is executing a given routine. It is to be understood that the housings, although described as being situated at the hips, behind the knees and/or at the ankles, the exact locations may be in the vicinity of these locations. For example, with respect to the housings at the hips, the locations of the housings may range from about the waistband to about halfway down towards the side of the knees. Similarly, in the transverse direction, the housings may be located anywhere from about the back of the leg to about the front of the leg in the vicinity of the hip (as defined above, for example). The size of the housings themselves may assume any size provided the sizes are large enough to house the electronic components. For example, the housings or pockets can be just large enough to receive the electronic components, or have spare space for receiving additional objects.

With respect to the housings disclosed to be located at the back of the knees, in some embodiments, it is to be understood that the exact locations of the housings may be in the vicinity of the back of the knees. For example, along the longitudinal axis or coronal plane of the smart yoga pants, the locations of the housings may range from about halfway up towards the hip to about halfway down towards the ankles, from about a third of the way up towards the hip to about a third of the way down towards the ankles, etc. Similarly, in the transverse direction, the housings may be located anywhere from about the one side of a knee to the other side of the.

With respect to the housings disclosed to be located at the ankles, in some embodiments, it is to be understood that the exact locations of the housings may be at any position in the vicinity of the ankles. For example, the housings may be anywhere along the circumference of the ankles, including in the inside of the ankles and/or the outside of the ankles. Further, along the longitudinal axis of the smart yoga pants/legs of a wearer, the housings may be in the range from about the bottom rim of the legs of the smart pants to about halfway, third of the ways, etc., up towards the knees.

Although the above discussions about the locations of the electronic components are presented with respect to housings or pockets, in some embodiments, the discussions equally apply when the electronic components are integrated within the fabrics of the smart yoga pants.

With reference to FIG. 2, in some embodiments, an example schematic illustrating the interplay of a smart garment worn by a user, an external device such as a smartphone used by the garment wearer to facilitate the execution of a physical activity, and an external server is shown. The garment wearer may be powered by onboard power sources 207 such as batteries. The smartphone 202 may include a user interface that, for example, allows the user to select a particular routine or pose so that the smartphone provides guidance on how to execute the routine or pose. For example, the smartphone may present on the user interface an image and/or video of the stance and/or movements that are to be executed to accomplish the routine or pose. Using smart yoga pants as a non-limiting example of a smart garment, for example, an application executing on the smartphone may present to the user one or more options 203, 204, 205 of yoga poses to select from for execution. Upon the activation of the electronic components of the pair of smart yoga pants worn by a user, in some embodiments, the yoga pants 201 and the smartphone 202 may begin to communicate with each other in one or more of communication protocols such as, but not limited to, the internet, Bluetooth, Bluetooth Low Energy (BLE), near field communication (NFC), Wi-Fi™, or any other radio frequency (RF) protocol. Initially, an application executing on the smartphone may allow a user (e.g., wearer of the yoga pants) to calibrate or synchronize the yoga pants 201 and the smartphone 202. The calibration may be configured to bring the sensors 207a and the actuators 207b (i.e., haptic motors) to a baseline and starting value. The application may avail to a user a variety of routines 203, 024, 205 the wearer of the pants may perform, and upon selection, instructions on how to perform the routine may appear in the application (on the user interface of the smartphone). The smart yoga pants 201 may communicate with the smartphone 202 and/or with the external server 206 via a communications circuit 207c. In some embodiments, the instructions may also be transmitted from the smartphone 202 to the wearer via the actuators 207b embedded in the smart pants 201. For example, upon selection of the Warrior II yoga routine, a series of steps may appear on the display of the smartphone (all at the same time or sequentially) depicting the poses that constitute the routine. In some instances, the instructions may be delivered to the user as an audio message (e.g., may be read out to the user via the smartphone's speaker). In addition to or instead of appearing on the display of the smartphone 202 or being read out by the speaker of the smartphone 202, the steps may also be communicated to the wearer via vibrations of the actuators 207b in the yoga pants. For example, a vibration of one or more actuators located in the vicinity of a knee may indicate to the wearer that a proper pose of the Warrior II routine includes the stacking of the knee over the ankle of the same leg. In some embodiments, the instructions may be delivered to the user using any number of the above-noted means. For example, the instructions may appear on the display of the smartphone 202, e.g., FIG. 3B, while at the same time the actuators in the smart yoga pants vibrate to deliver the same instructions. In some cases, the properties of the vibrations such as but not limited to patterns, frequency, intensity, etc., may represent different messages from the smartphone 202 to the wearer. For example, different patterns of vibrations may represent the progression of movements or poses of the routine, allowing for a more convenient identification of the instructions by the wearer of the smart pants.

In some embodiments, upon a wearer of the smart yoga pants performing a particular pose, a determination may be made by a processing unit 207e in the yoga pants and/or an external device such as the smartphone 202 (and/or the external server 206) on whether the wearer performed the routine/poses accurately, and if needed, corrective instructions may be sent to the smart yoga pants so as to correct the wearer's inaccurate poses or routine steps, as discussed in detail above. In some instances, the determination may be made once sensors 207a onboard the smart garment 201 have gathered all types of data with respect to the routine execution of the smart yoga pants wearer, and transmitted some or all of the data to the smartphone 202 and/or the external device 206. The corrective instructions may be transmitted to the smart garment 201 via the communications circuit 207c to instruct the actuators 207b to activate and vibrate in a manner that allows the user to understand the corrective instructions. Similar to above, the patterns, frequency, intensity, etc., of the vibrations may represent different instructions. For example, when a yoga pants wearer is executing a particular pose and the actuator at one of the knees is vibrating at different intensities, the different intensities may represent different amounts of angular offsets the user needs to execute at the knee to achieve the accurate pose.

Although the discussion above focused on yoga pants, it is to be understood that same or similar discussions apply to clothing articles worn for active physical exertions where the clothing articles come into contact, either directly or through other materials (underwear, for example), with the skins of wearers of the clothing articles. For example, the disclosures of the present application can apply to active wears worn while engaging in primarily “leg-specific” activities such as squats, jumping, lunges, etc. In some embodiments, the active wears may be clothing articles for other parts of the body; for example, shirts designed for body building activities.

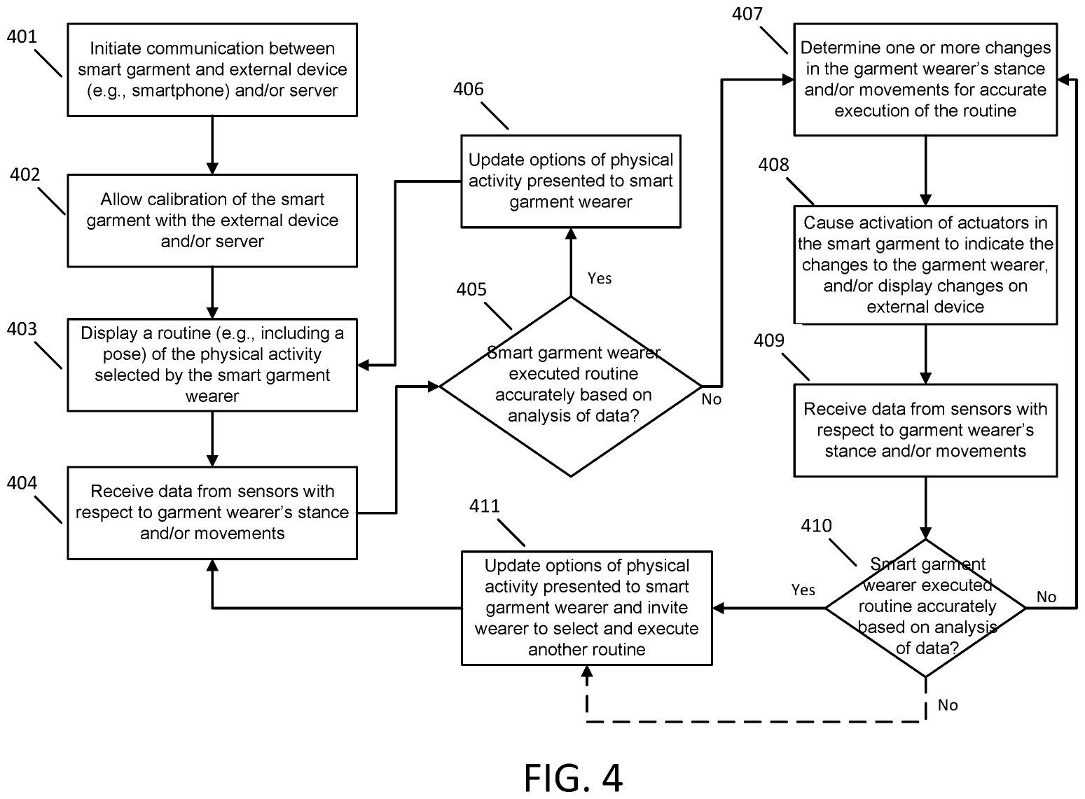

Example steps for calibrating and using the disclosed pair of smart garment according to the discussions above are shown in the flow diagram of FIG. 4. In FIG. 4, the execution of a physical activity routine by a wearer of a smart garment such as a smart pair of athletic yoga pants via control of actuators embedded in the yoga pants is shown, according to some embodiments. A smartphone with a wireless communications capability is linked with a pair of smart pants, e.g., 401, and a calibration or synchronization routine is executed to, amongst other things, bring the sensors and the actuators (i.e., haptic motors) embedded in the yoga pants to a baseline and starting value, e.g., 402. Once calibration is achieved, in some embodiments, a wearer or user of the smartphone may be allowed to choose a physical activity option (e.g., yoga poses) from one or more options displayed at the user interface of the smartphone, e.g., 403, and the wearer may receive instructions for guided routine via the smartphone and/or the actuators. Once the smart garment wearer attempts to execute the selected pose or routine, the sensors located throughout the smart garment may gather all relevant data (e.g., location, orientation, timestamps, etc.), and transmit the data to the smartphone and/or an external server for processing, e.g., 404. In some embodiments, the data may also be transmitted to a processing unit on the smart garment itself for processing. A processing unit (e.g., in the pair of yoga pants, the smartphone or some other internal or external processor operationally coupled to the yoga pants and/or the smartphone) may determine if the execution of the yoga pants wearer is acceptable, e.g., 405. The execution may comprise one or more yoga poses. In some embodiments, the processing unit may determine after each pose whether the wearer has performed the pose acceptably. In some embodiments, the processing unit may wait until after a series of poses have been performed before making such determinations (e.g., after a subroutine). If the smart yoga wearer has executed the routine or pose accurately within some predetermined or predefined tolerance (e.g., within the wearer's skill level or chosen level), the smartphone may acknowledge the user's effort as a success and update the options accordingly, e.g., 406. When the executions of the yoga pants wearer are below some determined standards, in some embodiments, the smartphone may prompt the wearer to repeat one or more poses or the entire routine so as to enhance the wearer's skills. In some embodiments, the processing unit may determine the changes that the wearer may execute to achieve a more accurate execution of the routine or the pose, e.g., 407, and generate corrective instructions accordingly to transmit to the smart garment to activate the actuators on the garment itself in a manner that allows the user know the messages of the corrective instructions, e.g., 408. In some instances, the same messages may be displayed on the user interface of the smartphone. Upon receiving data from the sensors on the user's attempt to comply with the messages as delivered by the vibrations and/or on the screen or user interface of the smartphone, e.g., 409, the processing unit may further analyze the data to establish the user's progress, e.g., 410. The analysis may result in the user being offered additional options of routines and poses to perform, e.g., 411, or repeat the just performed ones to improve the execution of same routines and/or poses. In some embodiments, the processing unit and/or the smartphone may generate a report of the wearer's execution of the routines after completion of the exercise.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be an example and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

At least some of the embodiments disclosed above, in particular at least some of the methods/processes disclosed, may be realized in circuitry, computer hardware, firmware, software, and combinations thereof (e.g., a computer system). Such computing systems, may include PCs (which may include one or more peripherals well known in the art), smartphones, specifically designed medical apparatuses/devices and/or other mobile/portable apparatuses/devices. In some embodiments, the computer systems are configured to include clients and servers. A client and server are generally remote from each other and typically interact through a communication network (e.g., VPN, Internet). The relationship of client and server arises by virtue of computer programs running on the respective computers and having a client-server relationship to each other.

Some embodiments of the disclosure (e.g., methods and processes disclosed above) may be embodied in a computer program(s)/instructions executable and/or interpretable on a processor, which may be coupled to other devices (e.g., input devices, and output devices/display) which communicate via wireless or wired connect (for example).

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

As noted elsewhere, the disclosed embodiments have been presented for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, compositions, systems and apparatuses/devices which may further include any and all elements from any other disclosed methods, compositions, systems, and devices, including any and all elements corresponding to detecting one or more target molecules (e.g., DNA, proteins, and/or components thereof). In other words, elements from one or another disclosed embodiments may be interchangeable with elements from other disclosed embodiments. Moreover, some further embodiments may be realized by combining one and/or another feature disclosed herein with methods, compositions, systems and devices, and one or more features thereof, disclosed in materials incorporated by reference.

In addition, one or more features/elements of disclosed embodiments may be removed and still result in patentable subject matter (and thus, resulting in yet more embodiments of the subject disclosure). Furthermore, some embodiments correspond to methods, compositions, systems, and devices which specifically lack one and/or another element, structure, and/or steps (as applicable), as compared to teachings of the prior art, and therefore represent patentable subject matter and are distinguishable therefrom (i.e. claims directed to such embodiments may contain negative limitations to note the lack of one or more features prior art teachings).

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What's claimed is:

1. A smart garment, comprising:
one or more sensors contained within the smart garment and configured to gather data on a stance and/or a movement of a wearer of the smart garment undertaking a physical activity; and
one or more actuators contained within the smart garment and configured to provide haptic feedback to the wearer of the smart garment; wherein:
the one or more sensors transmit the data to a processing unit for generation of instructions providing guidance on executing the stance and/or the movement; and
the one or more actuators provide haptic feedback to the wearer upon receiving the instructions from the processing unit.

2. The smart garment of claim 1, wherein the smart garment is a pair of yoga pants.

3. The smart garment of claim 1, wherein the one or more sensors comprise an accelerometer, and/or a gyro sensor.

4. The smart garment of claim 1, wherein the one or more sensors comprise at least two sensors, and the at least two sensors are configured to communicate with each other.

5. The smart garment of claim 1, further comprising the processing unit disposed within the smart garment.

6. The smart garment of claim 1, wherein the processing unit is disposed in a smartphone, and the instructions are displayed to the wearer via a user interface of the smartphone.

7. The smart garment of claim 1, further comprising: a power source for providing power to electrical components contained within the smart garment.

8. The smart garment of claim 1, further comprising: a communications circuit configured to facilitate communication amongst the one or more sensors, the one or more actuators and/or the processing unit.

9. The smart garment of claim 8, wherein the communications circuit is further configured to facilitate communication between electronic components contained within the smart garment and an external processor.

10. The smart garment of claim 1, wherein the one or more actuators are configured to vary one or more of an intensity, frequency and pattern of the haptic feedback based on content of the received instructions.

11. The smart garment of claim 1, wherein the data includes parameters related to relative positional relationship of the one or more sensors with respect to each other during an execution of the stance and/or the movement by the wearer.

12. The smart garment of claim 11, wherein the processing unit generates the instructions after comparing the parameters of the relative positional relationship of the one or more sensors to standard parameters representing accurate execution of the stance and/or the movement.

13. The smart garment of claim 12, wherein the parameters of the relative positional relationship include orientations of any two sensors with respect to each other.

14. The smart garment of claim 12, wherein the parameters of the relative positional relationship include distance between any two sensors with respect to each other.

15. The smart garment of claim 1, further comprising a timer for recording timestamp measurements during an execution of the stance and/or the movement by the wearer.

16. A system, comprising:
a smart garment including a sensor and an actuator, the sensor configured to collect data on a stance and/or a movement of a smart garment wearer and the actuator configured to provide haptic feedback to the smart garment wearer in response to an instructions message received from a processing unit; and
a communications circuit, operationally coupled to the sensor and the actuator, the communications circuit configured to transmit: (1) the data collected by the sensor to the processing unit, and (2) the instructions message generated by the processing unit to the actuator;
wherein:
the processing unit, upon receipt of the data from the communications circuit, is configured to:
identify a defining parameter of the stance and/or the movement of the garment wearer;
extract, from the transmitted information, a value representing the identified defining parameter of the stance and/or the movement; and
evaluate the extracted value for congruence with a standard value of the defining parameter of the stance and/or the movement, the standard value representing a correct execution of the stance and/or the movement.

17. The system of claim 16, wherein the sensor comprises a plurality of sensors, and the defining parameter includes orientation of any two sensors of the plurality of sensors with respect to each other.

18. The system of claim 16, wherein the sensor comprises a plurality of sensors, and the defining parameter includes distance between any two sensors of the plurality of sensors.

19. The system of claim 16, further comprising a timer for recording timestamp measurements during an execution of the stance and/or the movement by the wearer.

20. A method, comprising:
receiving, at a user interface of a computing device, a selection identifying a routine of a physical activity to be reenacted by a user wearing a smart garment, the routine characterized by a defining parameter;
receiving, at a processor of the computing device, data on a stance and/or a movement of the user reenacting the routine, the data collected by a sensor integrated into the smart garment;
extracting, from the received data, a value representing the defining parameter of the stance and/or the movement of the routine reenacted by the user; and
evaluating the extracted value for congruence with a standard value of the defining parameter of the routine, the standard value representing a correct execution of the routine.
